(19) United States
(12) Patent Application Publication
(10) Pub. No.: US 2005/0288587 A1
(43) Pub. Date: Dec. 29, 2005

(54) Drive machanism for mechanically scanned ultrasound transducers

(76) Inventors: Yongrae Roh, Daegu (KR); Sanghan Lee, Daegu (KR); Ohbum Kwon, Daegu (KR); Susung Lee, Daegu (KR); Kookjin Kang, Daegu (KR)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 10/877,868

(22) Filed: Jun. 25, 2004

(51) Int. Cl. A61B 8/00

(52) U.S. Cl. 600/445

(57) ABSTRACT

The size, weight, and shape of a wobbler transducer are more optimized by positioning a drive shaft of a motor orthogonal to an array rather than parallel with the array. The drive shaft may be more perpendicular than parallel to the direction of the transducer movement as well. Different devices may be used for transferring the force of the rotational movement of the motor to the array. For example, bevel gears are used to rotate a belt. The array connects with the belt for mechanical movement. As another example, an arm is rotated 180°. The arm slidingly and rotatably connects with the transducer array. As the arm rotates in response to the motor, the array is slid along a rail with bushings. A smaller size motor may be used as large torque may not be needed for driving the array as compared to a typical wobbler transducer. A more ergonomic probe housing, such as a smaller and more desirably shaped housing may be provided.

### BACKGROUND

The present invention relates to a drive mechanism for mechanically scanned ultrasound transducers.

Three- or four-dimensional ultrasonic images may assist in diagnosis. A three-dimensional volume is scanned electronically using a two- or a one-dimensional array electrically scanned along one dimension and mechanically scanned along another dimension. Arrays mechanically scanned along one dimension are wobbler arrays. A one-dimensional array is modified to be connected with a motor or other driving mechanism for mechanically scanning.

FIG. 1 shows one example of a known wobbler transducer 20. A linear array 22 is connected with a motor 26 by an arm 24. The motor 26 includes a drive shaft for driving reduction gearing 28. The reduction gearing connects with the arm 24 at a center of rotation 30. The rotational radius from the center 30 to the transducer array 22 should be large for linear or planar mechanical scanning. The large radius requires a large torque to move the array. To generate the large torque, a higher power motor is used. The reduction gearing 28 also assists in conversion of velocity to torque. The reduction gearing 28 also acts to slow movement of the transducer 22 to allow for a dense scan of a patient. The drive shaft of the motor 26 is positioned generally parallel with the array 22, resulting in an inconvenient positioning of the motor for handheld use by the user. The bulky motor and rigid metal frame for supporting the motor increase the weight. The size and weight result in a transducer probe that is inconvenient for gripping.

Different types of ultrasonic transducers are used for different imaging applications. For example, a convex array is used for imaging deep and/or wide organs, such as abdominal or obstetrical uses. A high frequency and compact array is used for organs which are small or close to skin tissues, such as the breast or carotid artery. In the abdominal use, mechanically scanning a larger region is achieved by rotating the array with a large rotational angle, such as represented by the arrow in FIG. 1. However, for diagnosing small parts such as the breast or carotid, more precise imaging information may be obtained by linear or planar mechanical scanning. The rotational structure of the wobbler transducer 20 prevents such linear or planar movement of the array 22.

### BRIEF SUMMARY

By way of introduction, the preferred embodiments described below include drive mechanisms for a mechanically scanned ultrasound transducer. The size, weight, and shape of a wobbler transducer are more optimized by positioning a drive shaft of a motor orthogonal to an array rather than parallel with the array. The drive shaft may be more perpendicular than parallel to the direction of the transducer movement as well. Different devices may be used for transferring the force of the rotational movement of the motor to the array. For example, bevel gears are used to rotate a belt. The array connects with the belt for mechanical movement. As another example, an arm is rotated 180°. The arm slidingly and rotatably connects with the transducer array. As the arm rotates in response to the motor, the array is slid along a rail with bushings. Other mechanisms may be used. A smaller size motor may be used as large torque may not be needed for driving the array as compared to the wobbler transducer 20 shown in FIG. 1. A more ergonomic probe housing, such as a smaller and more desirably shaped housing may be provided.

In one aspect, a drive mechanism for a mechanically scanned ultrasound transducer is provided. An array of elements is moveable substantially along a surface. The surface is one of a curved surface, a flat plane or combinations thereof. A motor has a drive shaft. The drive shaft is positioned more perpendicular than parallel to the surface. The drive shaft connects with the array of elements, and the motor is operable to move the array of elements substantially along the surface.

In a second aspect, a drive mechanism is provided for a mechanically scanned ultrasound transducer. A belt connects with a motor and an array of elements. An array of elements is operable to move in response to movement of the belt from force from the motor.

In a third aspect, a wobbler transducer is provided for three- or four-dimensional ultrasound imaging. An array of elements is moveable substantially along a surface. The drive shaft of a motor is positioned more perpendicular than parallel to the surface. A pulley connects with another drive shaft. A belt is connected with the pulley and the array of elements. The array of elements is operable to move in response to movement of the belt relative to the pulley.

In a fourth aspect, a drive mechanism is provided for a mechanically scanned ultrasound transducer. An arm connects with a motor and an array of elements. The array of elements is moveable along a surface in response to movement of the arm. The arm is moveable in response to force from the motor. The arm is moveable substantially parallel to the surface.

In a fifth aspect, a drive mechanism is provided for a mechanically scanned ultrasound transducer. A bushing connects with an array of elements and a rail. The rail and the bushing are operable to guide the array of elements during movement caused by a motor.

In a sixth aspect, a wobbler transducer for three- or four-dimensional ultrasound imaging is provided. An arm connects with a motor and an array of elements. The array of elements is moveable along a surface in response to movement of the arm. The arm is moveable in response to force from the motor, and the arm is moveable substantially parallel to the surface. A bushing connects with the array of elements and a rail. The rail and bushing are operable to guide the array of elements during movement caused by the motor.

The present invention is defined by the following claims, and nothing in this section should be taken as a limitation on those claims. Further aspects and advantages of the invention are discussed below in conjunction with the preferred embodiments and may be later claimed independently or in combination. Different embodiments of the present invention may or may not achieve any of the various advantages discussed herein.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a side view of a prior art wobbler transducer;

FIG. 2 is a perspective view of a first embodiment of a drive mechanism for mechanically scanning an ultrasound array;

FIG. 3 is a cross-section view of a second embodiment of a drive mechanism for mechanically scanning an ultrasound array;

FIG. 4 is a end view of some components of the drive mechanism of FIG. 2;

FIG. 5 is a partial cutaway view of some of the components of the drive mechanism of FIG. 2;

FIG. 6 is a side view of some components of the drive mechanism of FIG. 2;

FIG. 7 is a perspective view of some of the components of the drive mechanism of FIG. 3;

FIG. 8 is a perspective view of some of the components of the drive mechanism of FIG. 3;

FIG. 9 is a perspective view of other components of the drive mechanism of the drive mechanism of FIG. 3;

FIG. 10 is a side view of some of the components of the drive mechanism of FIG. 3; and

FIG. 11 is a graphical representation of velocity as a function of angular position of an array for the drive mechanism of FIG. 3.

### DETAILED DESCRIPTION OF THE DRAWINGS AND PRESENTLY PREFERRED EMBODIMENTS

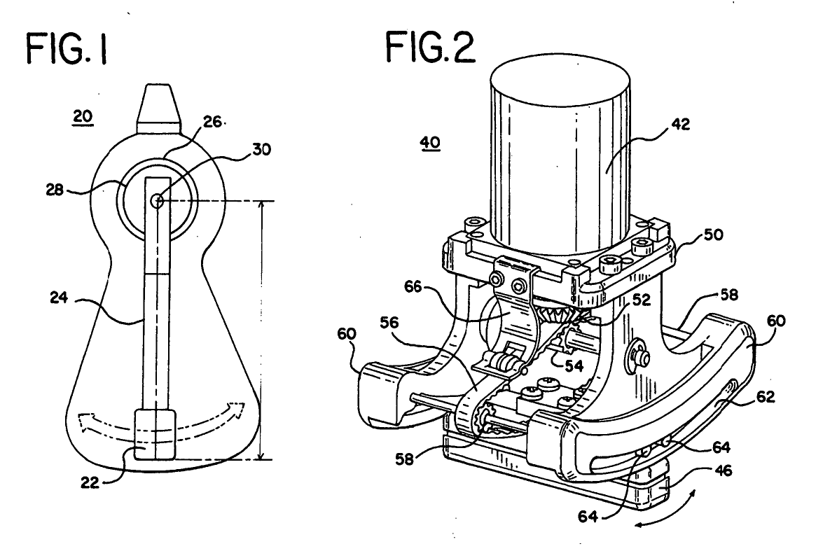

FIGS. 2 and 3 show two different embodiments of a drive mechanism for a mechanically scanned ultrasound transducer. Other drive mechanisms than shown in FIGS. 2 and 3 may be used. Components and arrangements of components common to both drive mechanisms of FIGS. 2 and 3 are discussed in general prior to discussing the components specific to each of the different drive mechanisms.

Each of the drive mechanism 40 shown in FIGS. 2 and 3 are associated with a motor 42 and associated drive shaft 44 positioned more perpendicular than parallel to an array of elements 46, the direction of mechanical movement of the array 46 and/or a surface of an effective array (i.e. the surface defined by the azimuth extent of the array and the elevational displacement of the array). Other motor 42 positions, such as more parallel, may be used.

The array 46 of elements is an array of two or more piezoelectric, capacitive membrane, microelectromechanical, combinations thereof or other elements operable to transduce between acoustical and electrical energies. In one embodiment, the array 46 is a one-dimensional linear, curved linear, convex or concave array. The elements extend in a single row along an azimuth dimension. In other embodiments, a 1.25, 1.5, 1.75 or 2-dimensional array of elements is provided. The array 46 may also include additional components, such as matching layers, backing block and/or electrodes.

The array 46 is moveable substantially along a surface. Substantially along is used to account for manufacturing tolerance based deviations from the desired surface. Substantially along may also account for movement along a curved surface being moved along a plane. The surface is any of a curved surface, a flat plane or combinations thereof. For example, a wobbler transducer shown in FIG. 1 moves the array 22 along a curved surface. The array 46 extends along one dimension of the surface, such as along the azimuth dimension of the array. The other dimension of the surface is defined by the path of movement of the transducer array 46. Using a one-dimensional array, the array 46 is moveable substantially along an elevation dimension generally perpendicular to the azimuth dimension. Alternatively, the array 46 is moved mechanically along the azimuth dimension or along any vector in a volume.

The array 46 is used to electronically scan along the azimuth dimension and mechanically scan along the elevation or other dimension. By scanning within a volume, a three-dimensional image may be generated. The repetitive rotational or linear movement of the one-dimensional array 46 may allow for four-dimensional imaging, three-dimensional imaging as a function of time.

The motor or 42 is a stepper motor which can control the angle of rotations of the drive shaft 44. Alternatively, the motor 42 is a magnetic, hydraulic, electric or other motor operational to generate rotational motion. The motor 42 is operable to provide 9.8 oz-in, but a greater or lesser torque may be provided. Given the general longitudinal shape of the motors, the reduced torque, and the positioning of the motor 42 discussed above, a housing 48 may be formed around the drive mechanism 40 with a convenient size, shape and weight for gripping by a user. The vertical positioning of the motor 42 more likely allows for a grip that is easily held by a user's hand that extends around the motor 42.

The drive shaft 44 is a metal rod, a rod of other materials, other structure for imparting rotational or longitudinal motion, combinations thereof or other now known or later developed drive shafts of a motor 42. The motor 42 and the associated drive shaft 44 are positioned to be more perpendicular than parallel to the surface of movement of the array 46. By activation of the motor 42, the drive shaft 44 rotates. The drive shaft 44 is connected with the array 46 of elements and the motor 42 to move the array 46 of elements. The connection is indirect or direct. For example, the drive shaft 44 directly connects with the motor 42 and indirectly connects with the array 46. Rotation of the drive shaft 44 is operable to move the array 46. The relative positioning of the drive shaft 44 and motor 42 to the array 46 may allow for the drive mechanism to be free of a reduction gear. In alternative embodiments, a reduction gear is provided. In yet other alternative embodiments, the motor 42 and/or the drive shaft 44 is positioned more parallel than perpendicular to the one- or two-dimensional surface formed by movement of the array 46.

FIG. 2 shows one embodiment of the drive mechanism 40. The drive mechanism 40 is used as a wobbler transducer for four- or three-dimensional ultrasound imaging. The drive mechanism 40 includes the motor 42, a frame 50, a bevel gear 52, a pulley 54, a belt 56, additional pulleys 58, guides 60, grooves 62, guide pins 64, the array 46, and a belt tensioner 66. Additional, different or fewer components may be provided, such as only one guide 60, only one pin 64, fewer or additional pulleys 58, or no belt tensioner 66.

The frame 50 is metallic, wood, fiberglass, plastic, combinations thereof or other now known or later developed materials. The frame 50 is formed as a one piece construction or from connecting together with glue, screws, bolts, combinations thereof or other connectors of multiple pieces. The frame 50 connects with the various components of the drive mechanism 40 for maintaining the relative positioning of the components.

The bevel gear 52 is fixedly mounted on the drive shaft 44 of the motor 42. The bevel gear 52 is anti-backlash bevel gear pinion, but may be formed of other types of bevel gears, gearing or pinions. The bevel gear 52 is fixedly mounted by bonding, pressure fit, barbs, bolts, set screws, lock washers or other components. The bevel gear 52 transfers the driving force from rotation of the drive shaft 44 to a different rotational axis. An u-joint or other structures for changing a rotational axis may alternatively be used.

The pulley 54 is a metal shaft or shaft of other materials. The shaft includes a timing gear for interaction with the belt 56 as well as a bevel gear 68. The pulley 54 is fixed in a rotational axis associated with the beveled gear 52. The bevel gear 68 is an anti-backlash bevel gear positioned on the pulley 54. The anti-backlash bevel gears 52 and 68 interconnect. The bevel gear 68 on the pulley 54 responds to the rotational force of the bevel gear 52 on the drive shaft 44 to cause rotation of the pulley 54. The rotational force of the motor 42 is transferred through the bevel gear 52 as a gear pinion to the bevel gear 68. Depending on the pitch, the pitch angle and processing quality, the bevel gears 52 and 68 may have a potential for backlash. Such backlash or reverse movement causes uncertainty in the precise angle or position of the array 46 for scanning, possibly distorting the resulting ultrasonic image. Anti-backlash bevel gears push gear teeth in a predetermined direction by a spring lock, preventing backlash or reverse movement of the gear.

Teeth along a portion of the pulley 54 in contact with the belt 56 or along the entire pulley 54 are used to transfer the motion of the pulley 54 to the belt 56 with minimal slipping. The teeth may prevent the belt 56 from slipping. In alternative embodiments, friction, a roughened surface, tension, surface texture, combinations thereof or other techniques are used for transferring force of rotation of the pulley 54 to the belt 56.

The additional pulleys 58 are of a same or different structure than the pulley 54. The additional pulleys 58 include teeth, grooves or other structure for maintaining a position of the belt 56 relative to the additional pulley 58. The additional pulleys 58 are spaced apart in order to distribute the belt 56 for desired movement of the array 46.

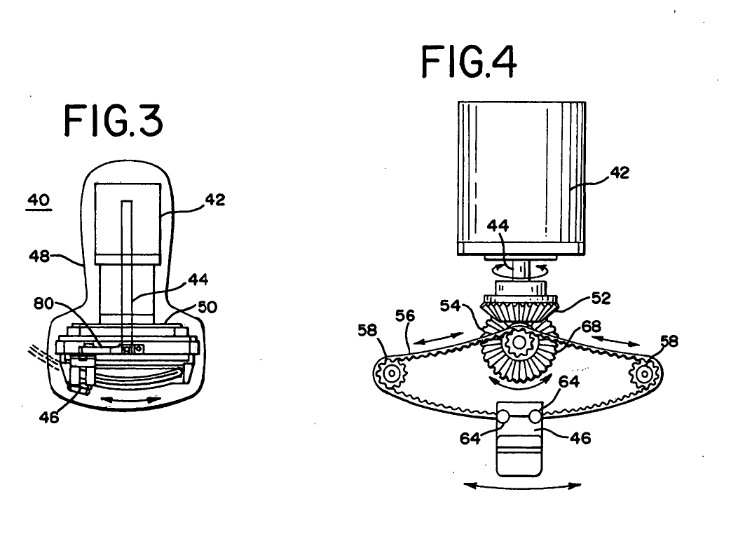

The belt 56 is an endless loop of rubber, plastic, fibers, combinations thereof or other now known or later developed belt materials. In one embodiment, the belt 56 is a closed loop timing belt with a high efficiency of kinetic power transmission from the timing pulley 54. The belt 56 includes a plurality of teeth on an inside of the loop for interacting with the pulley 54 and the additional pulleys 58. The top surface is flat, but may have other textures. The belt 56 extends over the pulleys 54, 58. In alternative embodiments, one or more additional pulleys 58 are positioned on an outside of the belt 56. Spacers, guides, or other structures may be used for guiding the belt 56 along a path shown in FIG. 4. The array 46 is mounted to the belt by a clamp, latch, bolts, screws, bonding, combinations thereof or other now known or later developed technique for attaching a structure to a belt 56.

The belt 56 connects with the motor 42 and the array 46. For example, the belt 56 connects with the motor through the pulley 54, and the array 46 is mounted to the belt 56 such that the array 46 is operable to move in response to movement of the belt 56 from force from the motor 42. The belt 56 moves relative to the pulley 54 by rotational force of the pulley 54, resulting in movement of the array 46. The diameter of the pulley 54 together with the bevel gears 52 and 68 and the speed of rotation of the drive shaft 44 determine the resolution of the array along the dimension of mechanical movement. The pulley 54 rotates a selected rotational angle per pulse of the motor 42. If the diameter of the pulley is large, the arc corresponding to a step angle by a single pulse of the motor 42 may be large. A large arc movement by the pulley 54 translates into a greater length of movement of the belt 56 across the pulley 54. Larger movement of the belt 56 translates into a larger movement the array 46. To maintain a high scanning density or resolution along the direction of mechanical movement, the pulley 54 is small so that the amount of movement of the belt 56 is short given the same amount of angular rotation of the pulley 54 and drive shaft 44. The sizing of the pulley 54 allows for control of the speed through design without a reduction gear. The size and power of the motor may also be less as compared to motors used for controlling the relatively large amounts of torque in the wobbler of FIG. 1.

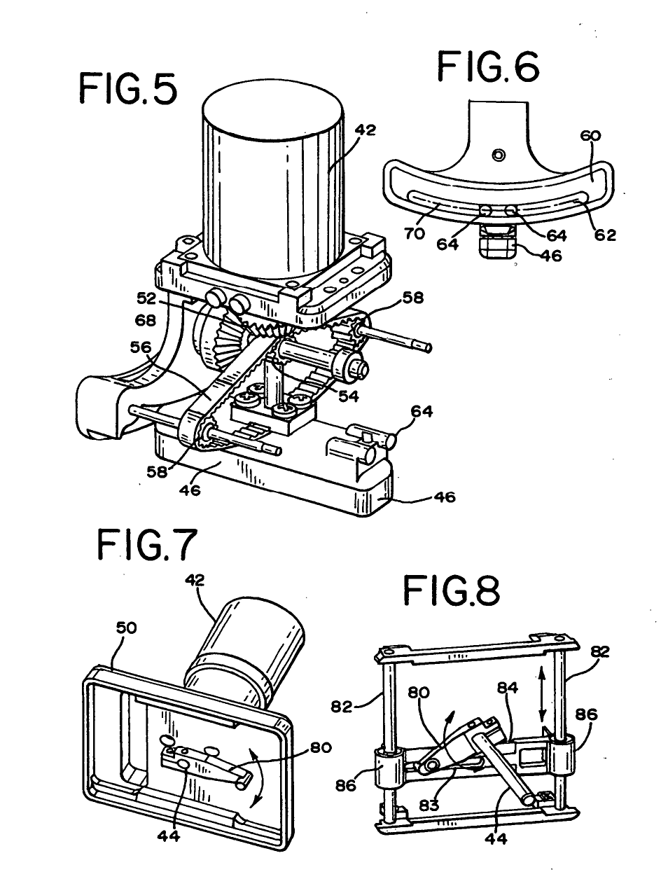

The guides 60 are formed as part of the frame 50 and include a groove 62 as shown in FIGS. 2 and 6. The array 46 includes pins 64 positionable within the groove 62 of the guide 60. Additional, different or fewer guides 60 may be provided. Two pins 64 per end of the array 46 are provided for restricting rotational movement of the array 46 relative to the guide 60. To avoid or minimize friction, plastics may be used for the pin 64 and/or the guide 60. Plastics having a sufficient mechanical rigidity, hardness and wear resistance are used, such as polyetherether ketone (PEEK) as the pin 64 and polyoxymethylene (POM) as the guide. Other friction reducing mechanisms may be used, such as oil or ball bearings. The pins 64 are inserted within the groove 62. As shown, the groove 62 extends through the guide 60, but may alternatively be an indention along one surface. The guide 60 is operable to guide the array 46 along the desired surface. For example, the pins 64 move only along a locus 70 of the groove 62.

The locus 70 and associated surface may be a flat plane or a curved surface. As shown in FIG. 6, a curved surface is provided for movement of the array 46. Curved surfaces provide a slight or some rotation 46 of the array throughout the path of the mechanical scan. A flat surface or linear locus 70 may prevent rotation of the array 46. While the guides 60 are shown positioned at the outer ends of the array 46, one or more guide 60 may be positioned further away from the center or closer to the center of the array 46.

The same general drive mechanism 40 may be used for scanning in both flat surfaces as well as curved surfaces. Different guides are available during assembly. Each of the guides has a different groove 62 and associated locus 70. The desired surface for mechanical scanning for a given drive mechanism 40 is selected during assembly. The associated guide 60 is selected for the assembly. The other parts of the drive mechanism 40 are common to both types of wobbler transducers.

The belt tensioner 66 shown in FIG. 2 is a roller connected with a leaf spring. The leaf spring is connected with the frame 50. The spring applies the tensioning force to the rollers. The rollers contact the belt 56, applying tension to the belt 56. Since the belt 56 may be subject to continuous tension and long use, the belt 56 may expand or loosen. A lose belt may result in less precise control of the array movement. The belt tensioner 66 provides for a more constant tension of the belt 56 despite age.

FIGS. 3 and 7-10 show another embodiment of a wobbler transducer for three- or four-dimensional ultrasound imaging. The wobbler transducer uses the drive mechanism 40 for mechanically scanning or moving the array 46 along at least one dimension. The drive mechanism 40 includes the motor or the combination of the motor and the gearhead 42 and associated drive shaft 44 oriented more perpendicular than parallel with the surface defined by the azimuth extent of the array 46 and the mechanical movement in the elevation dimension or other angle of the array 46. Rather than using a belt structure shown in FIG. 2, a rotating arm 80 is provided. The drive mechanism 40 also includes one or more rails 82, a slide 84, one or more bushings 86, guides 60, the array 46, the motor 42, the drive shaft 44, and the frame 50. Additional, different or fewer devices may be provided, such as providing the drive mechanism 40 without the guide 60 or with one or three or more rails 82 and associated bushings 86.

As shown in FIGS. 3, 7 and 8, an arm 80 connects with the drive shaft 44 of the motor 42 and the array 46 of elements. The arm 80 is metallic, plastic or other material for transmitting movement of the drive shaft 44 to the array 46. The connection is indirect or direct. For example, the arm 80 connects with the motor 42 through the drive shaft 44 and connects with the array of elements through the slide 84. The connection with the drive shaft 44 is a fixed connection, such as associated with bonding, a pressed fit, bolts, set screws, screws, latches, shaped tongue and groove, shaped shaft and hole, combinations thereof or other now known or later developed technique for preventing movement of the arm 40 different than or separate from the drive shaft 44 in at least one direction. The arm 80 rotatably connects with the array 46. For example and as shown in FIG. 8, the arm 80 includes a pin for insertion within a groove 83 of the slider 84. The pin at the end of the arm 80 is at a right angle to the arm 80 for interacting with the slide 84. As the arm 80 rotates with the drive shaft 40, the pin within the groove 83 slides and rotates within the groove 83. The change in position of the arm 80 causes the slider 84 to move along the rails 82. The arm 80 moves in a circle, such as over a 180° range. The arm 80 rotates in a plane substantially parallel to the surface of movement of the array 46 and/or parallel with the rails 82. The arm 80 has a length less than an azimuth extent of the array 46. For example, the arm 80 is less than half a length of the array 46 where the array 46 generally extends from one rail 82 to another rail 82. Greater or lesser length of the arm 80 or the array 46 may be used.

Each of the rails 82 are metal rods, but plastic or other materials may be used. The rails 82 are positioned within the frame 50 to guide movement of the slide 84 in response to rotation of the arm 80. The circular rotation of the arm 80 is transferred to a linear motion along the rails 82. As the arm moves back and forth over about a 180° or less range of rotation, the slide 84 moves back and forth along the rails 82.

The slide 84 includes bushings 86. The bushings 86 are linear bushings, such as bushings have a ball or a plurality of balls for rolling along the rails 82. As an alternative to balls, other reduced or low friction structures may be provided for sliding along the rails 82, such as a greased or oiled metal-to-metal contact, or Teflon coating. In response to the force from the arm 80 and the motor 42, the bushings 86 are slid along the rails 82. The array 46 is moved in response to or based on movement of the slider 84 and bushings 86. The rail 82 and bushings 86 guide the array 46 during movement.

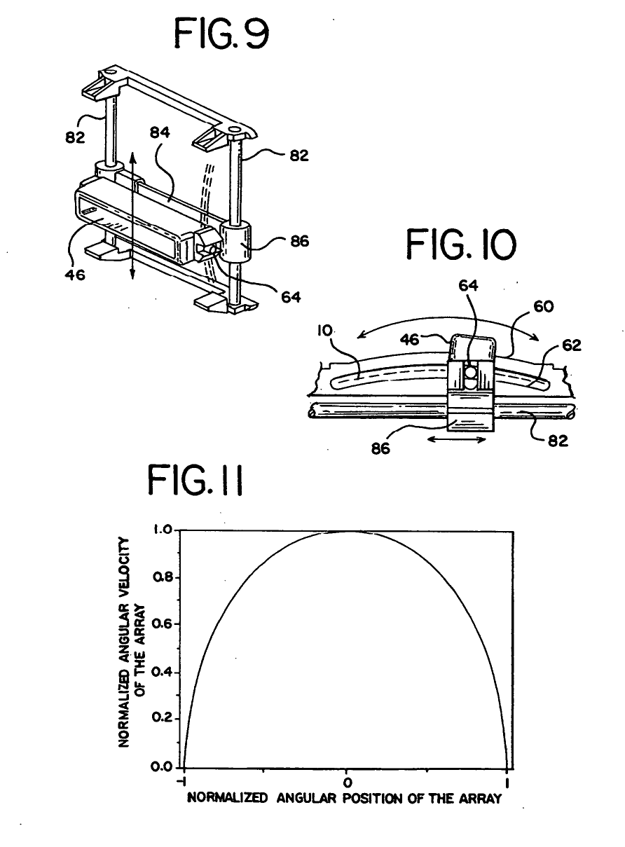

As shown in FIGS. 9 and 10, the array 46 includes one or more pins 64 at each end. The pin 64 is positioned within a groove of the slider 84 adjacent to the bushing 86. The guide 60 formed as part of the frame 50 includes the groove 62 and associated locus 70. In one embodiment, the radius of curvature of the groove 62 is 80 millimeters. As the bushing 86 traverses along the rail 82, the pin 64 is translated along the groove 62. Where the groove 62 is arced or otherwise curved, the pin 64 slides within the groove on the slider 84 for movement away from and towards the rail 82 during the lateral movement along or parallel with the rail 82. The bushing 86 slides along one dimension defined by the rail 82. The array 46 and slider 84 slide along the same dimension as the rail 82 and may also include motion perpendicular to the rail 82 as shown by the bold arrow in FIG. 10. The guide 60, groove 62 and pin 64 are operable to guide the array 46 along the desired surface. The guide 60 is separate than the rail 82 in one embodiment. In alternative embodiments, the rail 82 is used as a guide for scanning along a planar surface. In an alternative embodiment, the rail 82 is curved and used as a guide 60 without separate guide.

As the arm 80 is rotated over at about 180° range in a repetitive cycle, the array 46 reciprocally moves along or from one end to the other end of the rails 82 and associated groove 62. If the groove 62 has a curved configuration, the array 46 moves with an angular speed in a predetermined radius. To make resolution for a volume constant over a full or increased range of mechanical movement, intervals between the planes scanned at different mechanically placed positions of the array 46 are preferably constant. In order to move from the stationary array at one end to a stationary array at another end with an increased range of constant velocity, the speed of the motor 42 is accelerated or decelerated. If the angular speed of the arm 80 is constant, a velocity to position profile shown in FIG. 11 results. The normalized angular positions −1 and +1 correspond to a −90° and +90° position of the arm 80 (i.e. starting and ending positions). The array moves a shorter distance per pulse around the +1 and −1 positions than around the zero position. To make the profile more flat or associated with a more constant velocity as a function of position, the pulse frequency applied to the motor 42 is controlled. By modulating the pulse frequency applied to the motor 42, a greater acceleration and deceleration maybe provided at the ends of the extent of movement.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A drive mechanism for a mechanically scanned ultrasound transducer, the drive mechanism comprising:
an array of elements moveable substantially perpendicular to the array substantially along a surface comprising one of a curved surface, a flat plane or combinations thereof; and
a motor having a drive shaft, the drive shaft positioned more perpendicular than parallel to the surface;
wherein the drive shaft connects with the array of elements and the motor is operable to move the array of elements substantially along the surface.

2. The drive mechanism of claim 1 further comprising:
a pulley responsive to the drive shaft; and
a belt connected with the pulley and the array of elements, wherein the array of elements is operable to move in response to movement of the belt relative to the pulley.

3. The drive mechanism of claim 1 wherein the array of elements is a one-dimensional array of elements substantially along an azimuth dimension and wherein the array of elements is moveable substantially along an elevation dimension perpendicular to the azimuth dimension.

4. The drive mechanism of claim 1 wherein the drive shaft is operable to rotate for moving the array of elements.

5. The drive mechanism of claim 1 further comprising:
a first guide connected with the array of elements, the guide operable to guide the array of elements along the surface.

6. The drive mechanism of claim 5 further comprising a second guide available for assembly, the first guide different than the second guide, and both the first and second guides operable with the array of elements and with the motor.

7. The drive mechanism of claim 1 being free of a reduction gear.

8. The drive mechanism of claim 2 further comprising:
a first anti-backlash bevel gear on the drive shaft; and
a second anti-backlash bevel gear on the pulley, the first and second anti-backlash bevel gears interconnected.

9. The drive mechanism of claim 1 further comprising:
an arm connected with the drive shaft and rotatably connected with the array of elements, wherein rotation of the drive shaft is operable to rotate the arm and the arm is operable to move the array of elements.

10. The drive mechanism of claim 9 wherein the arm has a length less than an azimuth extent of the array of elements.

11. The drive mechanism of claim 1 further comprising:
a rail; and
a bushing connected with the array of elements, the bushing operable to move along the rail in response to force from the motor.

12. The drive mechanism of claim 11 further comprising:
a guide connected with the array of elements, the guide operable to guide the array of elements along the surface, the guide separate from the rail.

13. The drive mechanism of claim 9 further comprising:
a rail; and
a bushing connected with the array of elements, the bushing operable to move along the rail in response to force from the arm.

14. A drive mechanism for a mechanically scanned ultrasound transducer, the drive mechanism comprising:
an array of elements moveable substantially along a surface comprising one of a curved surface, a flat plane or combinations thereof; and
a motor having a drive shaft;
a pulley connected with the drive shaft by a gear; and
a belt connected with the pulley and the array of elements, wherein the array of elements is operable to move in response to movement of the belt from force from the motor.

15. The drive mechanism of claim 14 wherein the motor comprises a drive shaft connected with the array of elements, the drive shaft positioned more perpendicular than parallel to the surface.

16. The drive mechanism of claim 14 further comprising a bevel gear on the drive shaft, the drive shaft being non-parallel with an axis of rotation of the pulley.

17. A wobbler transducer for three or four dimensional ultrasound imaging, the wobbler transducer comprising:
an array of elements movable substantially along a surface; and
a motor having a drive shaft, the drive shaft positioned more perpendicular than parallel to the surface;
a pulley connected with the drive shaft; and
a belt connected with the pulley and the array of elements, wherein the array of elements is operable to move in response to movement of the belt relative to the pulley.

18. A drive mechanism for a mechanically scanned ultrasound transducer, the drive mechanism comprising:
an array of elements;
a motor; and
an arm connected with the motor and the array of elements, the array of elements being moveable along a surface in response to movement of the arm, the arm being moveable in response to force from the motor and the arm being moveable only within a plane substantially parallel to the surface.

19. The drive mechanism of claim 18 wherein the motor has a drive shaft, the drive shaft positioned more perpendicular than parallel to the surface, the arm fixedly connected to the drive shaft.

20. The drive mechanism of claim 18 further comprising:
a rail; and
a bushing connected with the array of elements and the rail, the rail and bushing operable to guide the array of elements during movement.

21. A drive mechanism for a mechanically scanned ultrasound transducer, the drive mechanism comprising:
an array of elements;
a motor;
a rail; and
a bushing connected with the array of elements and the rail, the rail and bushing operable to guide the array of elements during movement caused by the motor.

22. The drive mechanism of claim 21 wherein the array of elements is movable substantially along a surface; and
the motor has a drive shaft, the drive shaft positioned more perpendicular than parallel to the surface.

23. The drive mechanism of claim 21 further comprising:
an arm connected with the motor and the array of elements, the array of elements being moveable in response movement of the arm, the arm being moveable in response to force from the motor.

24. A wobbler transducer for three or four dimensional ultrasound imaging, the wobbler transducer comprising:
an array of elements;
a motor;
an arm connected with the motor and the array of elements, the array of elements being moveable along a surface in response to movement of the arm, the arm being moveable in response to force from the motor and the arm being moveable substantially parallel to the surface;
a rail; and
a bushing connected with the array of elements and the rail, the rail and bushing operable to guide the array of elements during movement caused by the motor.
